(19) United States
(12) Patent Application Publication
(10) Pub. No.: US 2019/0117052 A1
(43) Pub. Date: Apr. 25, 2019

(54) SURGICAL SYSTEM, SURGICAL CONTROL METHOD, AND PROGRAM

(71) Applicant: SONY CORPORATION, Tokyo (JP)

(72) Inventors: DAISUKE KIKUCHI, KANAGAWA (JP); TAKESHI UEMORI, TOKYO (JP); DAISUKE TSURU, CHIBA (JP); TAKESHI MIYAI, KANAGAWA (JP); KENTA YAMAGUCHI, KANAGAWA (JP)

(73) Assignee: SONY CORPORATION, Tokyo (JP)

(21) Appl. No.: 15/772,606

(22) PCT Filed: Oct. 31, 2016
(86) PCT No.: PCT/JP2016/082215

§ 371 Date: May 1, 2018

(30) Foreign Application Priority Data
Nov. 13, 2015 (JP) 2015-222898

(51) Int. Cl. A61B 1/045; A61B 1/07; G02B 23/26; G03B 35/08; G03B 15/05; H04N 5/232; H04N 5/235

(52) U.S. Cl. CPC A61B 1/045; A61B 1/07; G02B 23/26; G02B 7/36; G03B 15/05; H04N 5/232; H04N 5/235; G03B 35/08

(57) ABSTRACT

The present disclosure relates to a surgical system, a surgical control method, and a program enabling image processing with high accuracy in a case of changing an amount of light applied to a subject of a surgical imaging device. A light source control unit changes the light amount of the light applied to the subject imaged by an endoscope from a default value to a high light amount larger than the default value. An image processing unit performs image processing using a high light amount image which is an intraoperative image captured by the endoscope in a state in which the light amount is the high light amount. The image processing unit adjusts brightness of the high light amount image on the basis of the high light amount to generate a display image and displays the display image on a display device. The present disclosure is applicable to, for example, an endoscopic surgical system and the like.

### TECHNICAL FIELD

The present disclosure relates to a surgical system, a surgical control method, and a program, and especially relates to a surgical system, a surgical control method, and a program capable of performing image processing with high accuracy in a case of changing an amount of light applied to a subject of a surgical imaging device.

### BACKGROUND ART

Generally, in a surgical system which captures an intraoperative image with an endoscope, light of a constant light amount that is not insufficient in observation of a living body is continuously applied to the living body as a subject. Therefore, in a case where the light amount is large, damage to the living body due to heat and a power cost increase. Therefore, it is difficult to make the light amount equal to or larger than a predetermined amount.

Therefore, the endoscope which temporarily increases the amount of light applied to the subject is devised (refer to, for example, Patent Documents 1 and 2).

### CITATION LIST

### Patent Document

Patent Document 1: Japanese Patent Application Laid-Open No. 2006-149939

Patent Document 2: Japanese Patent Application Laid-Open No. 63-163809

### SUMMARY OF THE INVENTION

### Problems to be Solved by the Invention

However, in a case of changing an amount of light to be applied to a subject of a surgical imaging device such as an endoscope, it is not considered to perform image processing with high accuracy.

The present disclosure is achieved in view of such a situation, and an object thereof is to make it possible to perform the image processing with high accuracy in a case of changing the amount of light applied to the subject of the surgical imaging device.

### Solutions to Problems

A surgical system according to a first aspect of the present disclosure is a surgical system provided with a light source control unit that changes a light amount of light applied to a subject imaged by a surgical imaging device from a first light amount to a second light amount larger than the first light amount, an image processing unit that performs image processing using a high light amount image that is an intraoperative image captured by the surgical imaging device in a state in which the light amount is the second light amount, and a display control unit that adjusts brightness of the high light amount image on the basis of the second light amount to generate a display image and displays the display image on a display device.

A surgical control method and a program according to the first aspect of the present disclosure correspond to the surgical system according to the first aspect of the present disclosure.

In the first aspect of the present disclosure, a light amount of light applied to a subject imaged by a surgical imaging device is changed from a first light amount to a second light amount larger than the first light amount, image processing is performed using a high light amount image that is an intraoperative image captured by the surgical imaging device in a state in which the light amount is the second light amount, and brightness of the high light amount image is adjusted on the basis of the second light amount to generate a display image and the display image is displayed on a display device.

A surgical system according to a second aspect of the present disclosure is a surgical system provided with a light source control unit that changes a light amount of light applied to a subject of a surgical imaging device from a first light amount to a second light amount larger than the first light amount at a predetermined interval, and an image processing unit that generates a final intraoperative image by using a low light amount image that is an intraoperative image captured by the surgical imaging device when the light amount is the first light amount and a high light amount image that is an intraoperative image captured by the surgical imaging device when the light amount is changed to the second light amount.

In the second aspect of the present disclosure, a light amount of light applied to a subject of a surgical imaging device is changed from a first light amount to a second light amount larger than the first light amount at a predetermined interval, and a final intraoperative image is generated by using a low light amount image that is an intraoperative image captured by the surgical imaging device when the light amount is the first light amount and a high light amount image that is an intraoperative image captured by the surgical imaging device when the light amount is changed to the second light amount.

### Effects of the Invention

According to the first and second aspects of the present disclosure, it is possible to perform the image processing. Also, according to the first and second aspects of the present disclosure, it is possible to perform the image processing with high accuracy in a case of changing the amount of light applied to the subject of the surgical imaging device.

Note that the effects are not necessarily limited to the effects herein described and may include any of the effects described in the present disclosure.

### BRIEF DESCRIPTION OF DRAWINGS

FIG. 1 is a view illustrating a configuration example of a first embodiment of an endoscopic surgical system to which the present disclosure is applied.

FIG. 2 is a block diagram illustrating a configuration example of a CCU 12 in FIG. 1.

FIG. 3 is a view illustrating a first example of a light amount controlled by a light source control unit in FIG. 2.

FIG. 4 is a view illustrating a second example of the light amount controlled by the light source control unit in FIG. 2.

FIG. 5 is a block diagram illustrating a configuration example of an image processing unit in FIG. 2.

FIG. 6 is a flowchart illustrating image processing of the CCU in FIG. 2.

FIG. 7 is a block diagram illustrating a configuration example of an image processing unit 33 in a second embodiment of an endoscopic surgical system to which the present disclosure is applied.

FIG. 8 is a block diagram illustrating a configuration example of an image processing unit 33 in a third embodiment of an endoscopic surgical system to which the present disclosure is applied.

FIG. 9 is a block diagram illustrating a configuration example of an image processing unit 33 in a fourth embodiment of an endoscopic surgical system to which the present disclosure is applied.

FIG. 10 is a block diagram illustrating a configuration example of an image processing unit 33 in a fifth embodiment of an endoscopic surgical system to which the present disclosure is applied.

FIG. 11 is a view illustrating an example of a high light amount period and a low light amount period.

FIG. 12 is a flowchart illustrating image processing of a CCU in the fifth embodiment.

FIG. 13 is a block diagram illustrating a configuration example of hardware of a computer.

### MODE FOR CARRYING OUT THE INVENTION

A mode for carrying out the present disclosure (hereinafter, referred to as an embodiment) is hereinafter described. Note that the description is given in the following order.

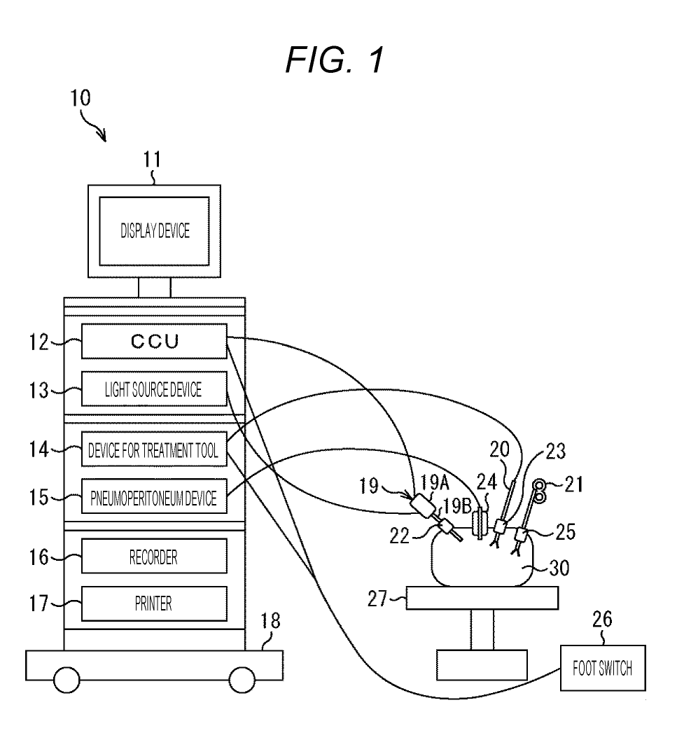

1. First Embodiment: Endoscopic Surgical System (FIGS. 1 to 6)

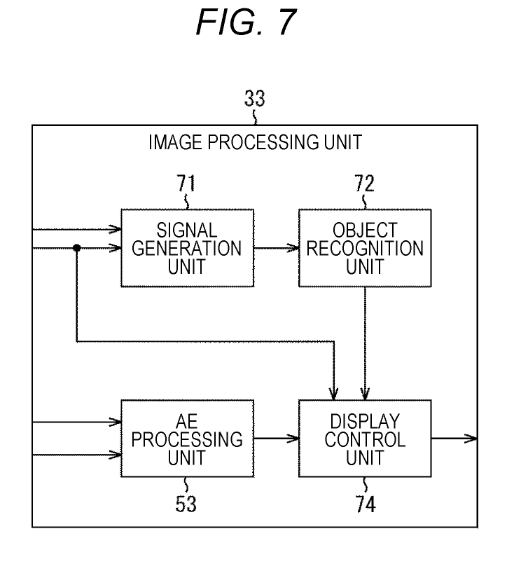

2. Second Embodiment: Endoscopic Surgical System (FIG. 7)

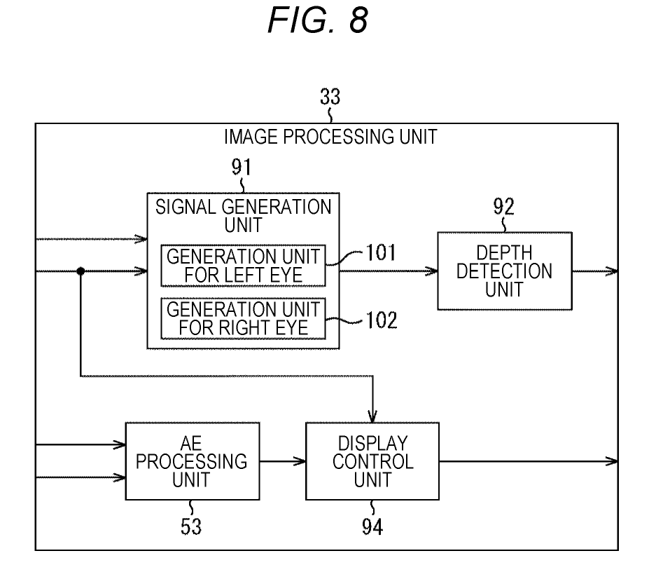

3. Third Embodiment: Endoscopic Surgical System (FIG. 8)

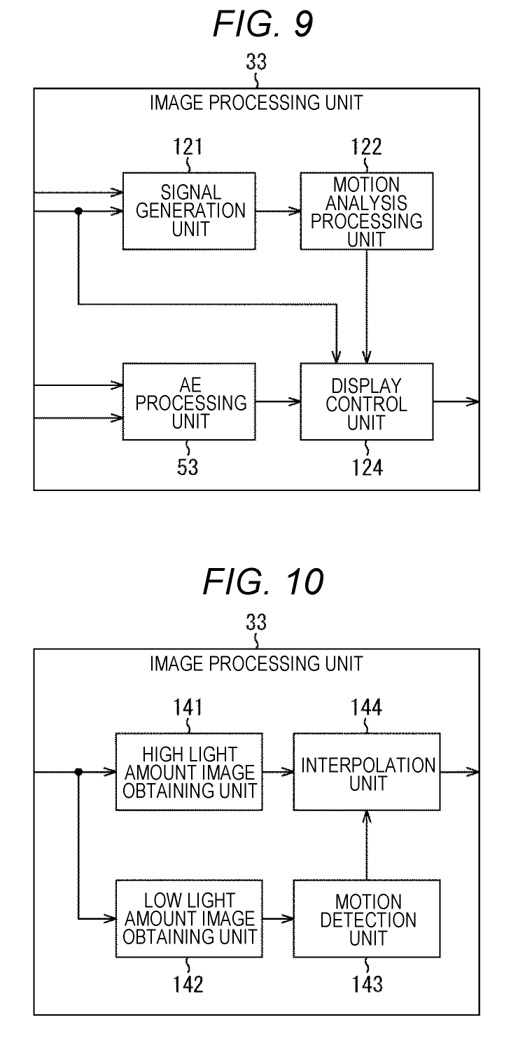

4. Fourth Embodiment: Endoscopic Surgical System (FIG. 9)

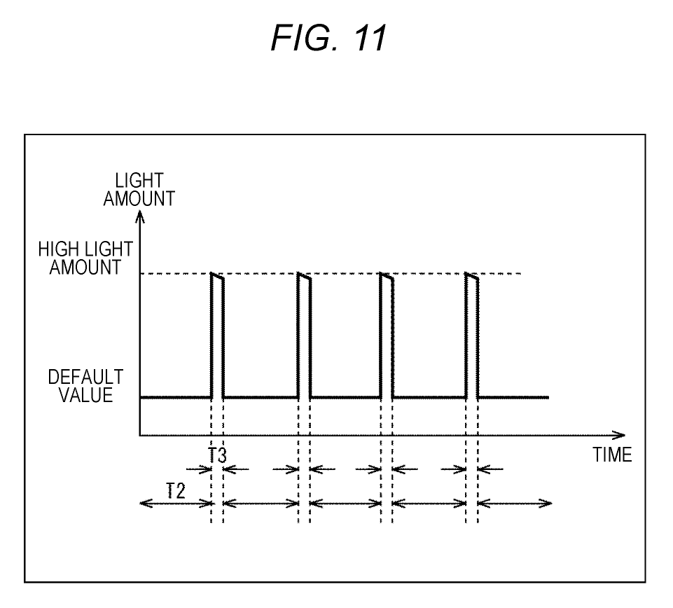

5. Fifth Embodiment: Endoscopic Surgical System (FIGS. 10 to 12)

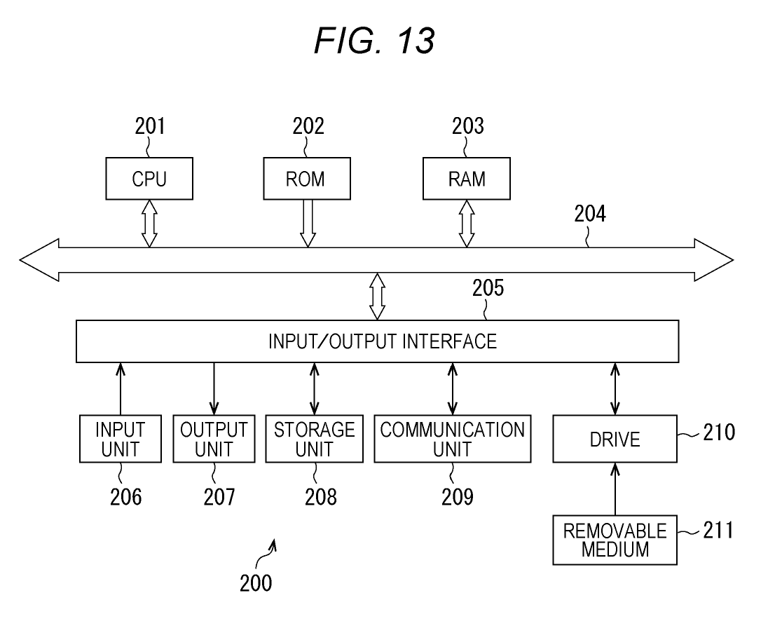

6. Sixth Embodiment: Computer (FIG. 13)

### First Embodiment

### Configuration Example of First Embodiment of Endoscopic Surgical System

FIG. 1 is a view illustrating a configuration example of a first embodiment of an endoscopic surgical system to which the present disclosure is applied.

An endoscopic surgical system 10 is provided with a cart 18 on which a display device 11, a camera control unit (CCU) 12, a light source device 13, a device for treatment tool 14, a pneumoperitoneum device 15, a recorder 16, and a printer 17 are mounted. The endoscopic surgical system 10 also includes an endoscope (laparoscope) 19, an energy treatment tool 20, forceps 21, trocars 22 to 25, a foot switch 26, and a patient bed 27. The endoscopic surgical system 10 arranged in, for example, an operating room assists an operator performing laparoscopic surgery on an affected site included in an abdomen 30 of a patient lying on the patient bed 27.

Specifically, the display device 11 of the endoscopic surgical system 10 includes a stationary 2D display, a head-mounted display and the like. The display device 11 displays an intraoperative image and the like supplied from the CCU 12.

The CCU 12 is connected to the endoscope 19 via a camera cable. Note that the CCU 12 may also be wirelessly connected to the endoscope 19. The CCU 12 receives the intraoperative image captured by the endoscope 19 and transmitted via the camera cable and supplies the same to the display device 11. The CCU 12 supplies the received intraoperative image to the recorder 16 and the printer 17 as needed.

Also, the CCU 12 (surgical system) controls an amount and a wavelength of light emitted from the light source device 13 on the basis of an operation signal and the like supplied from the foot switch 26. Furthermore, the CCU 12 performs image processing using the intraoperative image.

The light source device 13 is connected to the endoscope 19 via a light guide cable. Under the control of the CCU 12, the light source device 13 switches the amount and the wavelength of the emitted light. The light (for example, white light) emitted from the light source device 13 (light source unit) is applied to the inside of the abdomen 30 being a subject of the endoscope 19 via the light guide cable and the endoscope 19.

The device for treatment tool 14 being a high-frequency output device is connected to the energy treatment tool 20 and the foot switch 26 via cables. The device for treatment tool 14 outputs high-frequency current to the energy treatment tool 20 in response to the operation signal supplied from the foot switch 26.

The pneumoperitoneum device 15 provided with an air supply means and an air suction means supplies air into the abdomen 30 via a hole of the trocar 24 being a hole making tool attached to an abdominal wall of the abdomen 30.

The recorder 16 records the intraoperative image supplied from the CCU 12. The printer 17 prints the intraoperative image supplied from the CCU.

The endoscope 19 (surgical imaging device) includes a camera head 19A and a scope 19B. The camera head 19A is provided with an imaging unit such as a charge coupled device (CCD) and a complementary metal-oxide semiconductor (CMOS) sensor. The camera head 19A performs photoelectric conversion on the light from the inside of the abdomen 30 incident via the scope 19B and captures the intraoperative image being a moving image in frame units. The camera head 19A supplies the intraoperative image to the CCU 12 via the camera cable.

The scope 19B includes an optical system such as an illumination lens. The scope 19B is inserted into the abdomen 30 via a hole of the trocar 22 attached to the abdominal wall of the abdomen 30. The scope 19B irradiates the inside of the abdomen 30 with the light emitted from the light source device 13 and makes the light from the inside of the abdomen 30 to be incident on the camera head 19A.

Note that, although the camera head 19A is herein provided with the imaging unit, it is also possible that the scope 19B is provided with the imaging unit.

The energy treatment tool 20 includes an electric scalpel and the like. The energy treatment tool 20 is inserted into the abdomen 30 via a hole of the trocar 23 attached to the abdominal wall of the abdomen 30. The energy treatment tool 20 denatures or cuts the inside of the abdomen 30 using electric heat.

The forceps 21 are inserted into the abdomen 30 via a hole of the trocar 25 attached to the abdominal wall of the abdomen 30. The forceps 21 grasp the inside of the abdomen 30. The endoscope 19, the energy treatment tool 20, and the forceps 21 are grasped by the operator, an assistant, a scopist, a robot and the like.

The foot switch 26 accepts operation by a foot of the operator, the assistant and the like. The foot switch 26 supplies the operation signal indicating the accepted operation to the CCU 12 and the device for treatment tool 14.

By using the endoscopic surgical system 10 configured as described above, the operator may resect the affected site in the abdomen 30 without performing abdominal surgery in which the abdominal wall is cut to open the abdomen.

### Configuration Example of CCU

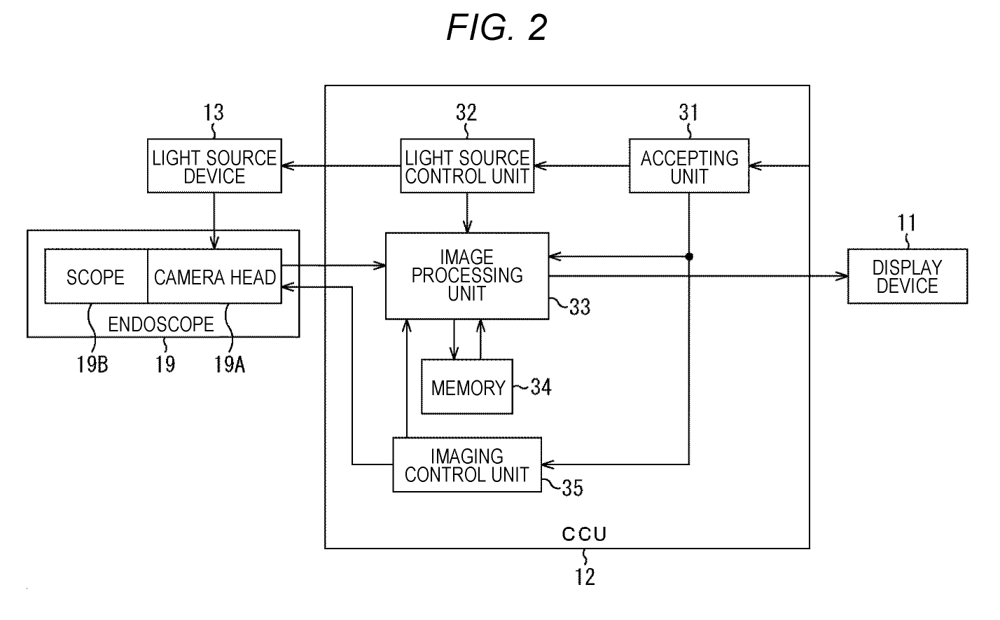

FIG. 2 is a block diagram illustrating a configuration example of the CCU 12 in FIG. 1.

The CCU 12 in FIG. 2 includes an accepting unit 31, a light source control unit 32, an image processing unit 33, a memory 34, and an imaging control unit 35.

The accepting unit 31 of the CCU 12 accepts operation by the operator and the like of the foot switch 26, an operation button not illustrated and the like. In response to the operation, the accepting unit 31 determines whether to perform auto focus (AF) control processing of the camera head 19A.

For example, when the accepting unit 31 accepts pressing by the operator and the like of an AF button not illustrated of the camera head 19A, this determines to perform the AF control processing. Note that the accepting unit 31 may determine to perform the AF control processing when accepting operation instructing to change zoom magnification, change a shooting mode, change the wavelength of the light emitted from the light source device 13 and the like.

Also, the accepting unit 31 may determine to perform the AF control processing in a case where a positional relationship between the subject and the endoscope 19 changes, or in a case where predetermined time elapses after last AF control processing instead of determining on the basis of the operation by the operator and the like.

In a case where the accepting unit 31 determines to perform the AF control processing of the camera head 19A, this instructs the light source control unit 32 to increase a light amount and instructs the image processing unit 33 and the imaging control unit 35 to start the AF control processing.

The light source control unit 32 changes the light amount of the light emitted from the light source device 13 from a default value (first light amount) to a light amount larger than the default value (second light amount) only in a case where the AF control processing is performed in response to the instruction to increase the light amount supplied from the accepting unit 31. When changing the light amount of the light emitted from the light source device 13 to a light amount larger than the default value (hereinafter, referred to as a high light amount), the light source control unit 32 notifies the image processing unit 33 of the high light amount. Also, the light source control unit 32 controls the wavelength of the light emitted from the light source device 13.

In response to the instruction to start the AF control processing supplied from the accepting unit 31, the image processing unit 33 performs various types of processing by using the intraoperative image captured when the light amount of the light emitted from the light source device 13 is the high light amount (hereinafter, referred to as a high light amount image) transmitted from the camera head 19A. For example, the image processing unit 33 adjusts brightness of the high light amount image on the basis of the high light amount supplied from the light source control unit 32 and an analog gain of the camera head 19A supplied from the imaging control unit 35 and transmits the high light amount image to the display device 11 to display. Also, the image processing unit 33 performs the AF control processing of the camera head 19A as image processing using the high light amount image.

Also, the image processing unit 33 transmits the intraoperative image captured when the light amount of the light emitted from the light source device 13 is the default value (hereinafter referred to as a low light amount image) transmitted from the camera head 19A to the display device 11 without change to display.

The high light amount image, the low light amount image, and an intermediate result of the AF control processing are stored in the memory 34 as necessary and are read by the image processing unit 33 at necessary timing.

The imaging control unit 35 controls the analog gain and a shutter speed of the camera head 19A in response to the instruction to start the AF control processing supplied from the accepting unit 31. Specifically, in a case where the instruction to start the AF control processing is supplied from the accepting unit 31, the light amount of the light emitted from the light source device 13 becomes the high light amount while the AF control processing is performed. Accordingly, during that time, the imaging control unit 35 decreases the analog gain of the camera head 19A and increases the shutter speed (shortens exposure time). The imaging control unit 35 supplies the analog gain of the camera head 19A to the image processing unit 33.

Note that it is possible that the imaging control unit 35 performs either one of the decrease in analog gain and the increase in shutter speed.

### First Example of Light Amount

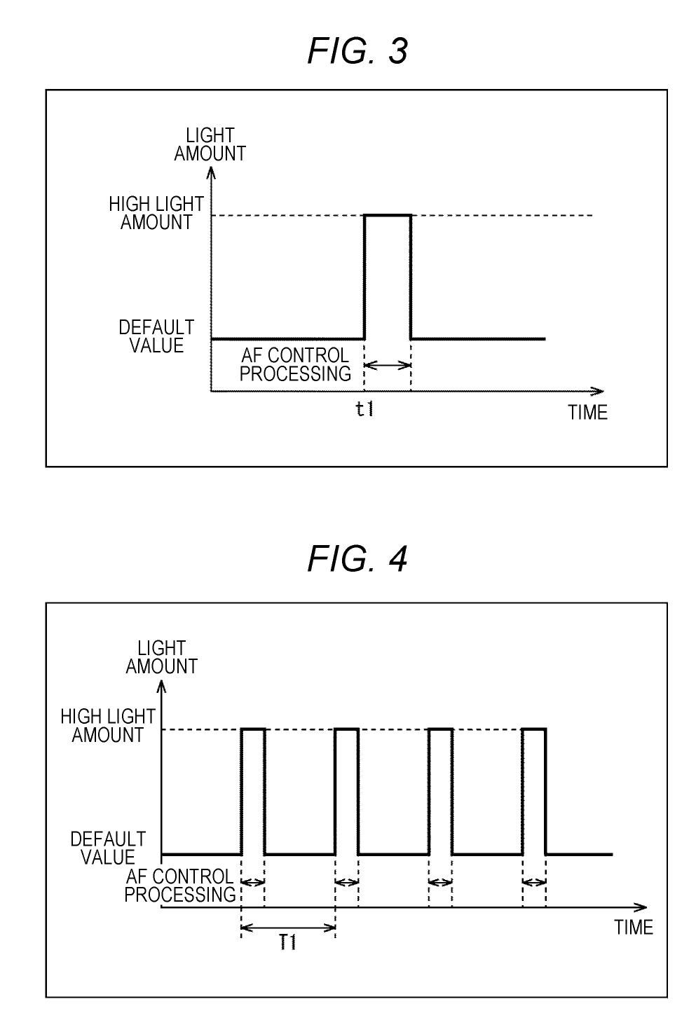

FIG. 3 is a view illustrating a first example of the light amount controlled by the light source control unit 32 in FIG. 2.

It should be noted that, in FIG. 3, time is plotted along the abscissa and the light amount of the light emitted from the light source device 13 is plotted along the ordinate. The same applies to FIGS. 4 and 11 to be described later.

In the example of FIG. 3, when accepting the operation from the operator and the like, the accepting unit 31 determines to perform the AF control processing with a trigger such as change in positional relationship between the subject and the endoscope 19.

In this case, as illustrated in FIG. 3, when the accepting unit 31 determines to perform the AF control processing at time t1, the light source control unit 32 changes the light amount of the light emitted from the light source device 13 from the default value to the high light amount only during a period in which the intraoperative image required for the AF control processing is captured in response to the instruction supplied from the accepting unit 31. In response to the instruction supplied from the accepting unit 31, the image processing unit 33 performs the AF control processing using only the high light amount image captured by the camera head 19A during this period.

### Second Example of Light Amount

FIG. 4 is a view illustrating a second example of the light amount controlled by the light source control unit 32 in FIG. 2.

In the example of FIG. 4, the accepting unit 31 determines to perform the AF control processing in a case where a certain period of time elapses after the last AF control processing, that is, to periodically perform the same.

In this case, as illustrated in FIG. 4, when the accepting unit 31 determines to perform the AF control processing at an interval of time T1, the light source control unit 32 changes the light amount of the light emitted from the light source device 13 from the default value to the high light amount only during the period in which the intraoperative image necessary for the AF control processing is captured in response the instruction supplied from the accepting unit 31. In response to the instruction supplied from the accepting unit 31, the image processing unit 33 performs the AF control processing using only the high light amount image captured by the camera head 19A during this period.

### Configuration Example of Image Processing Unit

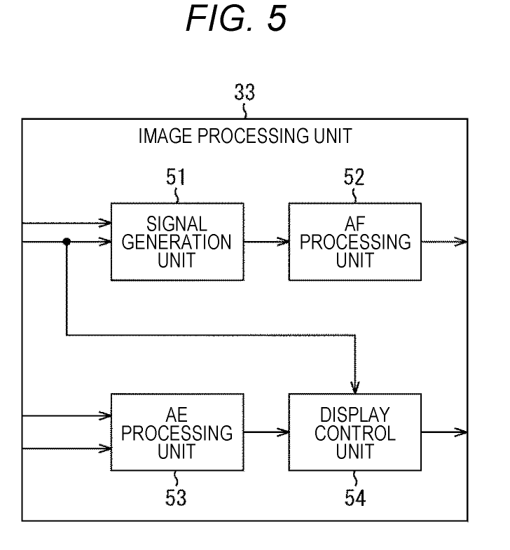

FIG. 5 is a block diagram illustrating a configuration example of the image processing unit 33 in FIG. 2.

The image processing unit 33 in FIG. 5 includes a signal generation unit 51, an AF processing unit 52, an AE processing unit 53, and a display control unit 54.

The signal generation unit 51 of the image processing unit 33 generates an image for AF from the high light amount image transmitted from the camera head 19A in response to the instruction to start the AF control processing supplied from the accepting unit 31 in FIG. 2. The image for AF is, for example, an image obtained by reducing resolution of the high light amount image. The signal generation unit 51 supplies the image for AF to the AF processing unit 52.

The AF processing unit 52 (image processing unit) performs the AF control processing of the camera head 19A using the image for AF supplied from the signal generation unit 51. Herein, the image for AF is generated from the high light amount image in which the analog gain of the camera head 19A is decreased and the shutter speed is increased by the imaging control unit 35. Therefore, the image for AF is the image in which noise is reduced by the decrease in analog gain and motion blur is reduced by the increase in shutter speed. Therefore, the AF processing unit 52 may perform the AF control processing with high accuracy by performing the AF control processing of the camera head 19A using the image for AF.

On the basis of the analog gain (imaging gain) supplied from the imaging control unit 35 in FIG. 2 and the light amount supplied from the light source control unit 32, the AE processing unit 53 determines an adjustment value of the brightness of the high light amount image such that the brightness of the high light amount image is the same as the brightness of the low light amount image. The AE processing unit 53 supplies the adjustment value of the brightness of the high light amount image to the display control unit 54.

On the basis of the adjustment value of the brightness supplied from the AE processing unit 53, the display control unit 54 adjusts the brightness (gain) of the high light amount image transmitted from the camera head 19A and transmits the high light amount image (display image) obtained as a result to the display device 11 in FIG. 2 to display on the display device 11. Also, the display control unit 54 transmits the low light amount image transmitted from the camera head 19A to the display device 11 without change to display. As described above, the brightness of the intraoperative image displayed on the display device 11 is constant regardless of the change in light amount of the light emitted from the light source device 13.

### Description of Image Processing of Endoscopic Surgical System

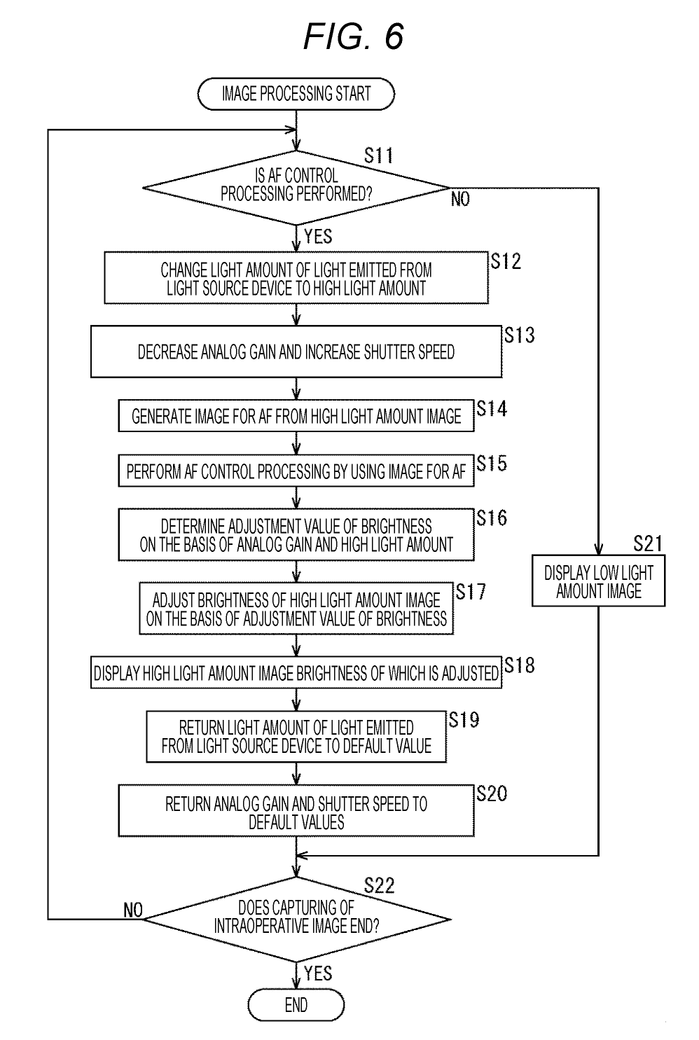

FIG. 6 is a flowchart illustrating the image processing of the CCU 12 of the endoscopic surgical system 10 in FIG. 1. This image processing starts, for example, when the capturing of the intraoperative image by the camera head 19A and the irradiation of the light by the light source device 13 are started.

At step S11 in FIG. 6, the accepting unit 31 (FIG. 2) of the CCU 12 determines whether to perform the AF control processing of the camera head 19A.

In a case where it is determined at step S11 to perform the AF control processing of the camera head 19A, the accepting unit 31 instructs the light source control unit 32 to increase the light amount and instructs the image processing unit 33 and the imaging control unit 35 to start the AF control processing. Then, at step S12, the light source control unit 32 changes the light amount of the light emitted from the light source device 13 from the default value to the high light amount and notifies the image processing unit 33 of the high light amount.

At step S13, the imaging control unit 35 decreases the analog gain of the camera head 19A from the default value and increases the shutter speed from the default value in response to the instruction from the accepting unit 31. The imaging control unit 35 supplies the decreased analog gain to the image processing unit 33.

At step S14, the signal generation unit 51 (FIG. 5) of the image processing unit 33 generates the image for AF from the high light amount image transmitted from the camera head 19A and supplies the same to the AF processing unit 52.

At step S15, the AF processing unit 52 performs the AF control processing using the image for AF supplied from the signal generation unit 51.

At step S16, on the basis of the analog gain supplied from the imaging control unit 35 and the high light amount supplied from the light source control unit 32, the AE processing unit 53 determines the adjustment value of the brightness of the high light amount image such that the brightness of the high light amount image is the same as the brightness of the low light amount image. The AE processing unit 53 supplies the adjustment value of the brightness of the high light amount image to the display control unit 54.

At step S17, the display control unit 54 adjusts the brightness of the high light amount image transmitted from the camera head 19A on the basis of the adjustment value of the brightness supplied from the AE processing unit 53. At step S18, the display control unit 54 transmits the high light amount image the brightness of which is adjusted to the display device 11 to display.

At step S19, the light source control unit 32 returns the light amount of the light emitted from the light source device 13 to the default value. At step S20, the imaging control unit 35 returns the analog gain and the shutter speed of the camera head 19A to the default values and shifts the procedure to step S22.

On the other hand, in a case where it is determined at step S11 that the AF control processing is not performed, at step S21, the image processing unit 33 transmits the low light amount image transmitted from the camera head 19A to the display device 11 without change to display. Then, the procedure shifts to step S22.

At step S22, the CCU 12 determines whether the capturing of the intraoperative image by the camera head 19A ends. In a case where it is determined at step S22 that the capturing of the intraoperative image does not end, the procedure returns to step S11 and the subsequent processes are repeated.

On the other hand, in a case where it is determined at step S22 that the capturing of the intraoperative image ends, the procedure ends.

As described above, the CCU 12 changes the light amount of the light emitted from the light source device 13 to the high light amount in a case where the AF control processing is performed, so that it is possible to perform the AF control processing using the high light amount image. Therefore, the CCU 12 may perform the AF control processing with higher accuracy as compared with a case where the AF control processing is performed using the low light amount image.

Also, the CCU 12 sets the light amount of the light emitted from the light source device 13 to the default value in a case where the AF control processing is not performed, so that it is possible to decrease an average light amount as compared with a case where the high light amount is always set. Therefore, power saving and cost reduction of the endoscopic surgical system may be realized. In addition, since an amount of heat generated by the light applied by the light source device 13 may be suppressed, damage to the abdomen 30 may be reduced.

Furthermore, the CCU 12 adjusts the brightness of the high light amount image so as to be equal to the brightness of the low light amount image on the basis of the high light amount, so that it is possible to prevent the brightness of the intraoperative image displayed on the display device 11 from being changed by the light amount of the light emitted from the light source device 13.

### Second Embodiment

### Configuration Example of Image Processing Unit in Second Embodiment of Endoscopic Surgical System

A configuration of a second embodiment of an endoscopic surgical system to which the present disclosure is applied is the same as a configuration in FIG. 1 except that object recognition processing of recognizing an object in an intraoperative image is performed as image processing in place of AF control processing.

Specifically, a configuration of a CCU in the second embodiment is the same as a configuration in FIG. 2 except that an accepting unit 31 determines whether to perform not the AF control processing but the object recognition processing, and except a configuration of an image processing unit 33.

The accepting unit 31 determines whether to perform the object recognition processing, for example, depending on whether pressing by an operator and the like of an object recognition button of a camera head 19A is accepted. Note that the accepting unit 31 may also determine whether to perform the object recognition processing depending on whether operation of instructing to change zoom magnification, change a shooting mode, change a wavelength of light emitted from a light source device 13 and the like is accepted just like the determination of whether to perform the AF control processing. Also, just like the determination of whether to perform the AF control processing, the accepting unit 31 may determine whether to perform the object recognition processing on the basis of change in positional relationship between a subject and an endoscope 19 and elapsed time from last object control processing.

FIG. 7 is a block diagram illustrating a configuration example of the image processing unit 33 in the second embodiment of the endoscopic surgical system to which the present disclosure is applied.

In the configuration illustrated in FIG. 7, the same reference sign is assigned to the same configuration as that in FIG. 5. Overlapping description is appropriately omitted.

The configuration of the image processing unit 33 in FIG. 7 differs from the configuration in FIG. 5 in that a signal generation unit 71, an object recognition unit 72, and a display control unit 74 are provided in place of a signal generation unit 51, an AF processing unit 52, and a display control unit 54. The image processing unit 33 in FIG. 7 performs not the AF control processing but the object recognition processing as image processing.

Specifically, the signal generation unit 71 of the image processing unit 33 generates an image for object recognition from a high light amount image transmitted from the camera head 19A in response to an instruction to start the object recognition processing supplied from the accepting unit 31. The signal generation unit 71 supplies the image for object recognition to the object recognition unit 72.

The object recognition unit 72 (image processing unit) performs the object recognition processing using the image for object recognition supplied from the signal generation unit 71. Specifically, the object recognition unit 72 performs matching processing between the image for object recognition and an image of a specific biological tissue or organ and recognizes the specific biological tissue or organ in the image for object recognition.

Herein, the image for object recognition is generated from the high light amount image in which an analog gain of the camera head 19A is decreased and a shutter speed thereof is increased by an imaging control unit 35. Therefore, the image for object recognition is an image in which noise is reduced by the decrease in analog gain and motion blur is reduced by the increase in shutter speed. Therefore, the object recognition unit 72 may perform the object recognition processing with high accuracy by performing the object recognition processing using the image for object recognition. The object recognition unit 72 supplies a result of the object recognition processing to the display control unit 74.

On the basis of an adjustment value of brightness supplied from an AE processing unit 53, the display control unit 74 adjusts brightness of the high light amount image transmitted from the camera head 19A and transmits the high light amount image obtained as a result to a display device 11 to display the same on the display device 11. Also, the display control unit 74 transmits a low light amount image transmitted from the camera head 19A to the display device 11 without change to display. As described above, brightness of the intraoperative image displayed on the display device 11 is constant regardless of change in light amount emitted from the light source device 13.

In addition, the display control unit 74 superimposes object information indicating an area of the recognized biological tissue or organ and the like on the high light amount image or the low light amount image being displayed on the display device 11 on the basis of the result of the object recognition processing supplied from the object recognition unit 72.

The image processing of the CCU 12 of the second embodiment is similar to image processing in FIG. 6 except that an image for AF is replaced by the image for object recognition, AF control processing is replaced by the object recognition processing, and the object information is superimposed on the high light amount image and the low light amount image, so that the description thereof is omitted.

The CCU 12 of the second embodiment changes the light amount of the light emitted from the light source device 13 to the high light amount in a case where the object recognition processing is performed, so that the object recognition processing may be performed using the high light amount image. Therefore, the CCU 12 may perform the object recognition processing with higher accuracy as compared with a case where the object recognition processing is performed using the low light amount image.

Note that although the image processing is the object recognition processing in the second embodiment, this may be object detection processing of detecting a specific biological tissue or organ present in the high light amount image. Also, the result of the object recognition processing may be supplied to another image processing unit not illustrated included in the CCU 12 and used for various types of image processing instead of being used by the display control unit 74.

### Third Embodiment

### Configuration Example of Image Processing Unit in Third Embodiment of Endoscopic Surgical System

A configuration of a third embodiment of an endoscopic surgical system to which the present disclosure is applied is the same as a configuration in FIG. 1 except that a camera head 19A includes two imaging units, depth detection processing of detecting a depth of an intraoperative image is performed as image processing in place of AF control processing, and a display device 11 performs 3D display.

Specifically, a configuration of a CCU in the third embodiment is the same as a configuration in FIG. 2 except that an accepting unit 31 determines whether to perform not the AF control processing but the depth detection processing, and except a configuration of an image processing unit 33.

The accepting unit 31 determines whether to perform the depth detection processing, for example, depending on whether pressing by an operator and the like of a depth detection button of the camera head 19A is accepted. Note that the accepting unit 31 may also determine whether to perform the depth detection processing depending on whether operation of instructing to change zoom magnification, change a shooting mode, change a wavelength of light emitted from a light source device 13 and the like is accepted just like the determination of whether to perform the AF control processing. Also, just like the determination of whether to perform the AF control processing, the accepting unit 31 may determine whether to perform the depth detection processing on the basis of change in positional relationship between a subject and an endoscope 19 and elapsed time from last depth detection processing.

FIG. 8 is a block diagram illustrating a configuration example of an image processing unit 33 in the third embodiment of the endoscopic surgical system to which the present disclosure is applied.

In the configuration illustrated in FIG. 8, the same reference sign is assigned to the same configuration as that in FIG. 5. Overlapping description is appropriately omitted.

The configuration of the image processing unit 33 in FIG. 8 differs from the configuration in FIG. 5 in that a signal generation unit 91, a depth detection unit 92, and a display control unit 94 are provided in place of a signal generation unit 51, an AF processing unit 52, and a display control unit 54. The image processing unit 33 in FIG. 8 performs not the AF control processing but the depth detection processing as image processing.

Specifically, the signal generation unit 91 of the image processing unit 33 includes a generation unit for left eye 101 and a generation unit for right eye 102. In response to an instruction to start the depth detection processing supplied from the accepting unit 31, the generation unit for left eye 101 obtains a high light amount image captured by the imaging unit on a left side facing the subject out of high light amount images of two viewpoints captured by the two imaging units transmitted from the camera head 19A. The generation unit for left eye 101 generates an image for depth detection from the obtained high light amount image and supplies the same to the depth detection unit 92 as an image for left eye.

In response to the instruction to start the depth detection processing supplied from the accepting unit 31, the generation unit for right eye 102 obtains the high light amount image captured by the imaging unit on a right side facing the subject out of the high light amount images of the two viewpoints transmitted from the camera head 19A. The generation unit for right eye 102 generates an image for depth detection from the obtained high light amount image and supplies the same to the depth detection unit 92 as an image for right eye.

The depth detection unit 92 (image processing unit) performs the depth detection processing using the image for left eye supplied from the generation unit for left eye 101 and the image for right eye supplied from the generation unit for right eye 102. Specifically, the depth detection unit 92 performs matching processing between the image for left eye and the image for right eye and detects a difference in position in the image of a pair of pixels having high similarity as the depth. This depth corresponds to a distance in a depth direction (optical axis direction) between the imaging unit and the subject.

Herein, the image for left eye and the image for right eye are generated from the high light amount image in which an analog gain of the camera head 19A is decreased and a shutter speed thereof is increased by an imaging control unit 35. Therefore, the image for left eye and the image for right eye are images in which noise is reduced by the decrease in analog gain and motion blur is reduced by the increase in shutter speed. Therefore, the depth detection unit 92 may perform the depth detection processing with high accuracy by performing the depth detection processing using the image for left eye and the image for right eye. The depth detection unit 92 supplies a depth map representing the depth of each pixel obtained as a result of the depth detection processing to the display device 11.

On the basis of an adjustment value of brightness supplied from an AE processing unit 53, the display control unit 94 adjusts brightness of the high light amount images of the two viewpoints transmitted from the camera head 19A and transmits the high light amount images obtained as a result to the display device 11. Also, the display control unit 94 transmits low light amount images of the two viewpoints transmitted from the camera head 19A to the display device 11 without change.

The display device 11 3D-displays the high light amount images or the low light amount images of the two viewpoints supplied from the display control unit 94 using the depth map supplied from the depth detection unit 92 as required. As described above, brightness of the intraoperative images of the two viewpoints 3D-displayed on the display device 11 is constant regardless of change in light amount emitted from the light source device 13.

Image processing of a CCU 12 of the third embodiment is similar to image processing in FIG. 6 except that an image for AF is replaced by the image for left eye and the image for right eye, AF control processing is replaced by the depth detection processing, and the high light amount images and the low light amount images are 3D-displayed on the basis of the result of the depth detection processing, so that the description thereof is omitted.

The CCU 12 of the third embodiment changes the light amount of the light emitted from the light source device 13 to the high light amount in a case where the depth detection processing is performed, so that the depth detection processing may be performed using the high light amount image. Therefore, the CCU 12 may perform the depth detection processing with higher accuracy as compared with a case where the depth detection processing is performed using the low light amount image.

Note that in the third embodiment, the display device 11 may also 2D-display the high light amount image or the low light amount image of one of the two viewpoints. Also, the depth map may be supplied to not the display device 11 but another image processing unit not illustrated included in the CCU 12 and used for various types of image processing.

### Fourth Embodiment

### Configuration Example of Image Processing Unit in Fourth Embodiment of Endoscopic Surgical System

A configuration of a fourth embodiment of an endoscopic surgical system to which the present disclosure is applied is the same as a configuration in FIG. 1 except that motion analysis processing of analyzing motion of a subject in an intraoperative image is performed as image processing in place of AF control processing.

Specifically, a configuration of a CCU in the fourth embodiment is the same as a configuration in FIG. 2 except that an accepting unit 31 determines whether to perform not the AF control processing but the motion analysis processing, and except a configuration of an image processing unit 33.

It is determined whether the accepting unit 31 performs the motion analysis processing, for example, depending on whether pressing by an operator and the like of a motion analysis button of a camera head 19A is accepted. Note that the accepting unit 31 may determine whether to perform the motion analysis processing depending on whether operation of instructing to change zoom magnification, change a shooting mode, change a wavelength of light emitted from a light source device 13 and the like is accepted just like the determination of whether to perform the AF control processing. Also, just like the determination of whether to perform the AF control processing, the accepting unit 31 may determine whether to perform the motion analysis processing on the basis of change in positional relationship between a subject and an endoscope 19 and elapsed time from last object control processing.

FIG. 9 is a block diagram illustrating a configuration example of the image processing unit 33 in the fourth embodiment of the endoscopic surgical system to which the present disclosure is applied.

In the configuration illustrated in FIG. 9, the same reference sign is assigned to the same configuration as that in FIG. 5. Overlapping description is appropriately omitted.

The configuration of the image processing unit 33 in FIG. 9 differs from the configuration in FIG. 5 in that a signal generation unit 121, a motion analysis processing unit 122, and a display control unit 124 are provided in place of a signal generation unit 51, an AF processing unit 52, and a display control unit 54. The image processing unit 33 in FIG. 9 performs not the AF control processing but the motion analysis processing as image processing.

Specifically, the signal generation unit 121 of the image processing unit 33 generates an image for motion analysis from a high light amount image transmitted from the camera head 19A in response to the instruction to start the motion analysis processing supplied from the accepting unit 31 and supplies the same to the motion analysis processing unit 122.

The motion analysis processing unit 122 holds the image for motion analysis supplied from the signal generation unit 121. The motion analysis processing unit 122 (image processing unit) performs the motion analysis processing using the held image for motion analysis of a past frame and the image for motion analysis of a current frame. Specifically, the motion analysis processing unit 122 performs block matching, a gradient method and the like using the past image for motion analysis and the current image for motion analysis, thereby detecting a motion vector of a subject included in the image for motion analysis to hold. The motion analysis processing unit 122 analyzes a period and the like of the motion vector on the basis of the motion vector of each frame.

Herein, the image for motion analysis is generated from a high light amount image in which an analog gain of the camera head 19A is decreased and a shutter speed thereof is increased by an imaging control unit 35. Therefore, the image for motion analysis is an image in which noise is reduced by the decrease in analog gain and motion blur is reduced by the increase in shutter speed. Therefore, the motion analysis processing unit 122 may perform the motion analysis processing with high accuracy by performing the motion analysis processing using the image for motion analysis. The motion analysis processing unit 122 supplies analysis information indicating the period and the like of the motion vector obtained as a result of the motion analysis processing to the display control unit 124.

On the basis of an adjustment value of brightness supplied from an AE processing unit 53, the display control unit 124 adjusts brightness of the high light amount image transmitted from the camera head 19A and transmits the high light amount image obtained as a result to a display device 11 to display the same on the display device 11. Also, the display control unit 124 transmits a low light amount image transmitted from the camera head 19A to the display device 11 without change to display. As described above, brightness of the intraoperative image displayed on the display device 11 is constant regardless of change in light amount emitted from the light source device 13.

In addition, the display control unit 124 superimposes period information indicating an area of the subject with the same period of the motion vector and the like on the high light amount image or the low light amount image being displayed on the display device 11 on the basis of the analysis information supplied from the motion analysis processing unit 122.

The image processing of the CCU 12 of the fourth embodiment is similar to image processing in FIG. 6 except that an image for AF is replaced by the image for motion analysis, the AF control processing is replaced by the motion analysis processing, and the period information is superimposed on the high light amount image and the low light amount image, so that the description thereof is omitted.

The CCU 12 of the fourth embodiment changes the light amount of the light emitted from the light source device 13 to the high light amount in a case where the motion analysis processing is performed, so that the motion analysis processing may be performed using the high light amount image. Therefore, the CCU 12 may perform the motion analysis processing with higher accuracy as compared with a case where the motion analysis processing is performed using the low light amount image.

Note that in the fourth embodiment, it is also possible that the analysis information is not used in the display control unit 74 but is supplied to another image processing unit not illustrated of the CCU 12 to be used in the various types of image processing.

### Fifth Embodiment

### Configuration Example of Image Processing Unit in Fifth Embodiment of Endoscopic Surgical System

A configuration of a fifth embodiment of an endoscopic surgical system to which the present disclosure is applied is the same as a configuration in FIG. 1 except that a light source control unit 32 changes a light amount of light emitted from a light source device 13 from a default value to a high light amount only during a period of one or more frames (hereinafter, referred to as a high light amount period) shorter than an interval at the interval of a period of a predetermined number of frames (hereinafter, referred to as a low light amount period), and except an image processing unit 33.

Specifically, a configuration of a CCU in the fifth embodiment is the same as a configuration in FIG. 2 except that the light source control unit 32 changes the light amount of the light emitted from the light source device 13 from the default value to the high light amount only during the high light amount period at the interval of the low light amount period, and an accepting unit 31 is not provided, and except the image processing unit 33.

FIG. 10 is a block diagram illustrating a configuration example of the image processing unit 33 in the fifth embodiment of the endoscopic surgical system to which the present disclosure is applied.

The image processing unit 33 in FIG. 10 includes a high light amount image obtaining unit 141, a low light amount image obtaining unit 142, a motion detection unit 143, and an interpolation unit 144.

The high light amount image obtaining unit 141 of the image processing unit 33 obtains a high light amount image captured by the camera head 19A during the high light amount period to be transmitted and supplies the same to the interpolation unit 144.

The low light amount image obtaining unit 142 obtains a low light amount image captured by the camera head 19A during the low light amount period to be transmitted and supplies the same to the motion detection unit 143.

The motion detection unit 143 holds the low light amount image supplied from the low light amount image obtaining unit 142. The motion detection unit 143 detects a motion vector of a subject using the held low light amount image of a last frame and the low light amount image of a current frame for each frame. The motion detection unit 143 supplies the motion vector of each frame to the interpolation unit 144.

The interpolation unit 144 outputs the high light amount image supplied from the high light amount image obtaining unit 141 as a final intraoperative image to a display device 11 to display and holds the same. The interpolation unit 144 performs motion compensation on the held high light amount image on the basis of the motion vector supplied from the motion detection unit 143 using a joint bilateral filter, a guided filter and the like, thereby generating an interpolation image for interpolating the high light amount image of the frame of the low light amount image. The interpolation unit 144 outputs the interpolation image as the final intraoperative image to the display device 11 to display. As described above, the final intraoperative image is equivalent to the high light amount image of all the frames.

### Example of High Light Amount Period and Low Light Amount Period

FIG. 11 is a view illustrating an example of the high light amount period and the low light amount period.

As illustrated in FIG. 11, in the fifth embodiment, the light source control unit 32 change the light amount of the light emitted from the light source device 13 from the default value to the high light amount only during a high light amount period T3 of one or more frames shorter than an interval T2 at the interval of the low light amount period T2.

Therefore, the number of frames of the high light amount image is smaller than that of the low light amount image. That is, resolution in a time direction of the high light amount image is lower than that of the low light amount image. However, the high light amount image is the intraoperative image in which an analog gain of a camera head 19A is decreased and a shutter speed thereof is increased by an imaging control unit 35, a high-definition image in which noise and motion blur are reduced.

On the other hand, the number of frames of the low light amount image is larger than that of the high light amount image. That is, the resolution in the time direction of the low light amount image is higher than that of the high light amount image. However, this is a low-definition image with much noise and motion blur.

Therefore, in the fifth embodiment, the motion detection unit 143 detects the motion vector of each frame using the low light amount image having high resolution in the time direction, and the interpolation unit 144 performs the motion compensation on the high-definition high light amount image on the basis of the motion vector. As a result, a high-definition and high-frame rate high light amount image is generated.

### Description of Image Processing of Endoscopic Surgical System

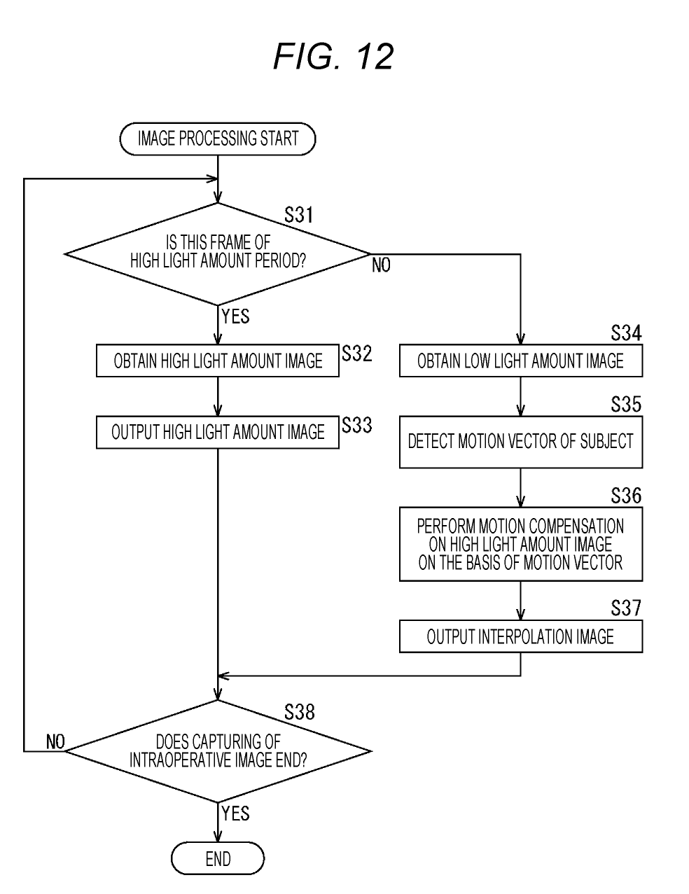

FIG. 12 is a flowchart illustrating image processing of a CCU 12 in the fifth embodiment. This image processing starts, for example, when the capturing of the intraoperative image by the camera head 19A and the irradiation of the light by the light source device 13 are started.

At step S31 in FIG. 12, the high light amount image obtaining unit 141 (FIG. 10) of the image processing unit 33 determines whether the current frame is the frame of the high light amount period. In a case where it is determined at step S31 that the current frame is the frame of the high light amount period, the high light amount image obtaining unit 141 obtains the high light amount image captured by the camera head 19A to be transmitted at step S32. The high light amount image obtaining unit 141 supplies the high light amount image to the interpolation unit 144.

At step S33, the interpolation unit 144 holds the high light amount image supplied from the high light amount image obtaining unit 141 and outputs the same without change as the final intraoperative image to the display device 11 to display. Then, the procedure shifts to step S38.

On the other hand, in a case where it is determined at step S31 that the current frame is not the frame of the high light amount period, the low light amount image obtaining unit 142 obtains the low light amount image captured by the camera head 19A to be transmitted at step S34. The high light amount image obtaining unit 141 supplies the low light amount image to the motion detection unit 143, and the motion detection unit 143 holds the low light amount image.

The motion detection unit 143 detects the motion vector of the subject using the held low light amount image of the last frame and the low light amount image of the current frame and supplies the same to the interpolation unit 144 at step S35.

At step S36, the interpolation unit 144 performs the motion compensation on the held high light amount image on the basis of the motion vector supplied from the motion detection unit 143 and generates the interpolation image of the frame of the low light amount image corresponding to the motion vector.

The interpolation unit 144 outputs the interpolation image as the final intraoperative image to the display device 11 to display at step S37. Then, the procedure shifts to step S38.

At step S38, the CCU 12 determines whether the capturing of the intraoperative image by the camera head 19A ends. In a case where it is determined at step S38 that the capturing of the intraoperative image does not end, the procedure returns to step S31 and subsequent processes are repeated.

On the other hand, in a case where it is determined at step S38 that the capturing of the intraoperative image ends, the procedure ends.

As described above, in the fifth embodiment, the motion detection unit 143 detects the motion vector of each frame using the low light amount image having the high resolution in the time direction, and the interpolation unit 144 performs the motion compensation on the high-definition high light amount image on the basis of the motion vector, thereby performing the interpolation. Therefore, the interpolation with higher accuracy may be performed as compared with a case where the interpolation is performed using only the high light amount images. In addition, it is possible to generate a higher-definition interpolation image as compared with a case where the interpolation is performed using only the low light amount image.

As a result, it is possible to allow the display device 11 to display the intraoperative image of all the frames equivalent to a case where the light amount of the light emitted from the light source device 13 is always the high light amount. Therefore, brightness of the intraoperative image displayed on the display device 11 is constant.

Also, the light source control unit 32 changes the light amount of the light emitted from the light source device 13 from the default value to the high light amount only in the high light amount period in the fifth embodiment, so that it is possible to decrease an average light amount as compared with a case where it is always set to the high light amount. Therefore, power saving and cost reduction of the endoscopic surgical system may be realized. In addition, since an amount of heat generated by the light applied by the light source device 13 may be suppressed, damage to the abdomen 30 may be reduced.

### Sixth Embodiment

### Description of Computer to Which Present Technology Is Applied

A series of processes of a CCU 12 described above may be executed by hardware or by software. In a case where a series of processes is performed by the software, a program which forms the software is installed on a computer. Herein, the computer includes a computer built in dedicated hardware, a general-purpose personal computer, for example, capable of executing various functions by various programs installed and the like.

FIG. 13 is a block diagram illustrating a configuration example of the hardware of the computer which executes the above-described series of processes of the CCU 12 by the program.

In a computer 200, a central processing unit (CPU) 201, a read only memory (ROM) 202, and a random-access memory (RAM) 203 are connected to one another by a bus 204.

An input/output interface 205 is further connected to the bus 204. An input unit 206, an output unit 207, a storage unit 208, a communication unit 209, and a drive 210 are connected to the input/output interface 205.

The input unit 206 includes a keyboard, a mouse, a microphone and the like. The output unit 207 includes a display, a speaker and the like. The storage unit 208 includes a hard disk, a non-volatile memory and the like. The communication unit 209 includes a network interface and the like. The drive 210 drives a removable medium 211 such as a magnetic disk, an optical disk, a magnetooptical disk, or a semiconductor memory.

In the computer 200 configured in the above-described manner, the CPU 201 loads the program stored in the storage unit 208, for example, on the RAM 203 via the input/output interface 205 and the bus 204 to execute, so that a series of processes described above is performed.

The program executed by the computer 200 (CPU 201) may be recorded on the removable medium 211 as a package medium and the like to be provided, for example. Also, the program may be provided by means of a wired or wireless transmission medium such as a local area network, the Internet, and digital broadcasting.

In the computer 200, the program may be installed on the storage unit 208 via the input/output interface 205 by mounting the removable medium 211 on the drive 210. Also, the program may be received by the communication unit 209 via the wired or wireless transmission medium to be installed on the storage unit 208. In addition, the program may be installed in advance on the ROM 202 and the storage unit 208.

Note that the program executed by the computer 200 may be the program of which processes are performed in time series in the order described in this specification or may be the program of which processes are performed in parallel or at required timing such as when a call is issued.

Also, in this specification, a system is intended to mean assembly of a plurality of components (devices, modules (parts) and the like) and it does not matter whether all the components are in the same casing. Therefore, a plurality of devices stored in different casings connected via the network and one device obtained by storing a plurality of modules in one casing are the systems.

The effect described in this specification is illustrative only and is not limitative; there may also be another effect.

Also, the embodiment of the present disclosure is not limited to the above-described embodiments and various modifications may be made without departing from the gist of the present disclosure.

For example, the image processing by the image processing unit 33 may be processing other than the AF control processing, the object recognition processing, the depth detection processing, the motion analysis processing, and the interpolation processing, and the image processing unit 33 may perform a plurality of types of image processing. In addition, the number of types of the light amount may be three or more. Furthermore, in the first to fourth embodiments, the light source device 13 may emit the light of the high light amount during a period other than the period in which the intraoperative image necessary for the image processing is captured.

Note that the present disclosure may also have the following configurations.

(1)

A surgical system provided with:

a light source control unit that changes a light amount of light applied to a subject imaged by a surgical imaging device from a first light amount to a second light amount larger than the first light amount;

an image processing unit that performs image processing using a high light amount image that is an intraoperative image captured by the surgical imaging device in a state in which the light amount is the second light amount; and

a display control unit that adjusts brightness of the high light amount image on the basis of the second light amount to generate a display image and displays the display image on a display device.

(2)

The surgical system according to (1) described above,

in which the light source control unit is configured to change the light amount to the second light amount only in a case where the image processing is performed.

(3)

The surgical system according to (1) or (2) described above,

in which the surgical imaging device is configured to decrease an imaging gain when the light amount is changed to the second light amount.

(4)

The surgical system according to (3) described above,

in which the display control unit is configured to adjust the brightness of the high light amount image on the basis of the second light amount and the imaging gain.

(5)

The surgical system according to any one of (1) to (4) described above,

in which the surgical imaging device is configured to shorten exposure time when the light amount is changed to the second light amount.

(6)

The surgical system according to any one of (1) to (5) described above,

in which the image processing unit is configured to perform focus control processing of controlling focus of the surgical imaging device using the high light amount image.

(7)

The surgical system according to any one of (1) to (5) described above,

in which the image processing unit is configured to perform object recognition processing of recognizing an object in the high light amount image using the high light amount image.

(8)

The surgical system according to any one of (1) to (5) described above,

in which the image processing unit is configured to perform depth detection processing of detecting a depth of the high light amount image using the high light amount image.

(9)

The surgical system according to any one of (1) to (5) described above,

in which the image processing unit is configured to perform motion analysis processing of analyzing motion of a subject in the high light amount image using the high light amount image.

(10)

The surgical system according to any one of (1) to (9) described above, further provided with:

a light source unit that irradiates the subject with light.

A surgical control method provided with:

a light source control step of changing a light amount of light applied to a subject imaged by a surgical imaging device from a first light amount to a second light amount larger than the first light amount;

an image processing step of performing image processing using a high light amount image that is an intraoperative image captured by the surgical imaging device in a state in which the light amount is the second light amount; and

a display control step of adjusting brightness of the high light amount image on the basis of the second light amount to generate a display image and displaying the display image on a display device

of a surgical system.

(12)

A program which allows a computer to serve as:

a light source control unit that changes a light amount of light applied to a subject imaged by a surgical imaging device from a first light amount to a second light amount larger than the first light amount;

an image processing unit that performs image processing using a high light amount image that is an intraoperative image captured by the surgical imaging device in a state in which the light amount is the second light amount; and

a display control unit that adjusts brightness of the high light amount image on the basis of the second light amount to generate a display image and displays the display image on a display device.

(13)

A surgical system provided with:

a light source control unit that changes a light amount of light applied to a subject of a surgical imaging device from a first light amount to a second light amount larger than the first light amount at a predetermined interval; and

an image processing unit that generates a final intraoperative image by using a low light amount image that is an intraoperative image captured by the surgical imaging device when the light amount is the first light amount and a high light amount image that is an intraoperative image captured by the surgical imaging device when the light amount is changed to the second light amount.

(14)

The surgical system according to (13) described above,

in which the light source control unit is configured to change the light amount to the second light amount only during a period shorter than the predetermined interval.

(15)

The surgical system according to (13) or (14) described above,

in which the image processing unit is provided with

a motion detection unit that detects motion of the subject using the low light amount image, and

an interpolation unit that generates an interpolation image for interpolating the high light amount image by performing motion compensation on the high light amount image on the basis of the motion detected by the motion detection unit and outputs the interpolation image and the high light amount image as the final intraoperative image.

(16)

The surgical system according to any one of (13) to (15) described above, further provided with:

a light source unit that irradiates the subject with light.

(17)

A surgical control method provided with:

a light source control step of changing a light amount of light applied to a subject of a surgical imaging device from a first light amount to a second light amount larger than the first light amount at a predetermined interval; and

an image processing step of generating a final intraoperative image by using a low light amount image that is an intraoperative image captured by the surgical imaging device when the light amount is the first light amount and a high light amount image that is an intraoperative image captured by the surgical imaging device when the light amount is changed to the second light amount

of a surgical system.

(18)

A program which allows a computer to serve as:

a light source control unit that changes a light amount of light applied to a subject of a surgical imaging device from a first light amount to a second light amount larger than the first light amount at a predetermined interval; and

an image processing unit that generates a final intraoperative image by using a low light amount image that is an intraoperative image captured by the surgical imaging device when the light amount is the first light amount and a high light amount image that is an intraoperative image captured by the surgical imaging device when the light amount is changed to the second light amount.

### REFERENCE SIGNS LIST

10 Endoscopic surgical system

11 Display device

12 CCU

13 Light source device

19 Endoscope

32 Light source control unit

33 Image processing unit

52 AF processing unit

54 Display control unit

144 Interpolation unit

1. A surgical system comprising:
a light source control unit that changes a light amount of light applied to a subject imaged by a surgical imaging device from a first light amount to a second light amount larger than the first light amount;
an image processing unit that performs image processing using a high light amount image that is an intraoperative image captured by the surgical imaging device in a state in which the light amount is the second light amount; and
a display control unit that adjusts brightness of the high light amount image on the basis of the second light amount to generate a display image and displays the display image on a display device.

2. The surgical system according to claim 1,
wherein the light source control unit is configured to change the light amount to the second light amount only in a case where the image processing is performed.

3. The surgical system according to claim 1,
wherein the surgical imaging device is configured to decrease an imaging gain when the light amount is changed to the second light amount.

4. The surgical system according to claim 3,
wherein the display control unit is configured to adjust the brightness of the high light amount image on the basis of the second light amount and the imaging gain.

5. The surgical system according to claim 1,
wherein the surgical imaging device is configured to shorten exposure time when the light amount is changed to the second light amount.

6. The surgical system according to claim 1,
wherein the image processing unit is configured to perform focus control processing of controlling focus of the surgical imaging device using the high light amount image.

7. The surgical system according to claim 1,
wherein the image processing unit is configured to perform object recognition processing of recognizing an object in the high light amount image using the high light amount image.

8. The surgical system according to claim 1,
wherein the image processing unit is configured to perform depth detection processing of detecting a depth of the high light amount image using the high light amount image.

9. The surgical system according to claim 1,
wherein the image processing unit is configured to perform motion analysis processing of analyzing motion of a subject in the high light amount image using the high light amount image.

10. The surgical system according to claim 1, further comprising:
a light source unit that irradiates the subject with light.

11. A surgical control method comprising:
a light source control step of changing a light amount of light applied to a subject imaged by a surgical imaging device from a first light amount to a second light amount larger than the first light amount;
an image processing step of performing image processing using a high light amount image that is an intraoperative image captured by the surgical imaging device in a state in which the light amount is the second light amount; and
a display control step of adjusting brightness of the high light amount image on the basis of the second light amount to generate a display image and displaying the display image on a display device
of a surgical system.

12. A program which allows a computer to serve as:
a light source control unit that changes a light amount of light applied to a subject imaged by a surgical imaging device from a first light amount to a second light amount larger than the first light amount;
an image processing unit that performs image processing using a high light amount image that is an intraoperative image captured by the surgical imaging device in a state in which the light amount is the second light amount; and
a display control unit that adjusts brightness of the high light amount image on the basis of the second light amount to generate a display image and displays the display image on a display device.

13. A surgical system comprising:
a light source control unit that changes a light amount of light applied to a subject of a surgical imaging device from a first light amount to a second light amount larger than the first light amount at a predetermined interval; and
an image processing unit that generates a final intraoperative image by using a low light amount image that is an intraoperative image captured by the surgical imaging device when the light amount is the first light amount and a high light amount image that is an intraoperative image captured by the surgical imaging device when the light amount is changed to the second light amount.

14. The surgical system according to claim 13,
wherein the light source control unit is configured to change the light amount to the second light amount only during a period shorter than the predetermined interval.

15. The surgical system according to claim 13,
wherein the image processing unit is provided with
a motion detection unit that detects motion of the subject using the low light amount image, and
an interpolation unit that generates an interpolation image for interpolating the high light amount image by performing motion compensation on the high light amount image on the basis of the motion detected by the motion detection unit and outputs the interpolation image and the high light amount image as the final intraoperative image.

16. The surgical system according to claim 13, further comprising:
a light source unit that irradiates the subject with light.

17. A surgical control method comprising:
a light source control step of changing a light amount of light applied to a subject of a surgical imaging device from a first light amount to a second light amount larger than the first light amount at a predetermined interval; and
an image processing step of generating a final intraoperative image by using a low light amount image that is an intraoperative image captured by the surgical imaging device when the light amount is the first light amount and a high light amount image that is an intraoperative image captured by the surgical imaging device when the light amount is changed to the second light amount
of a surgical system.

18. A program which allows a computer to serve as:
a light source control unit that changes a light amount of light applied to a subject of a surgical imaging device from a first light amount to a second light amount larger than the first light amount at a predetermined interval; and
an image processing unit that generates a final intraoperative image by using a low light amount image that is an intraoperative image captured by the surgical imaging device when the light amount is the first light amount and a high light amount image that is an intraoperative image captured by the surgical imaging device when the light amount is changed to the second light amount.
